(19) United States
(12) Patent Application Publication
(10) Pub. No.: US 2011/0077469 A1
(43) Pub. Date: Mar. 31, 2011

(54) SYSTEMS AND METHODS FOR UTILIZING PROLONGED SELF MONITORING IN THE ANALYSIS OF CHRONIC AILMENT TREATMENTS

(76) Inventors: Richard A. Blocker, Slidell, LA (US); Patricia L. Knaack, Slidell, LA (US)

(21) Appl. No.: 12/567,791

(22) Filed: Sep. 27, 2009

(51) Int. Cl. A61B 5/00

(52) U.S. Cl. 600/300

(57) ABSTRACT

A system that advantageously utilizes an individual's self-monitoring abilities in the analysis of various ailment treatments. This system may include a personal computing device that includes one or more processors, one or more memories, and a user interface for obtaining personal health data from a user of the personal computing device; as well as one or more biometric measurement devices that are capable of acquiring biometric measurement data. The personal computing device may be configured to receive various biodata and then store the biodata in memory. The processor(s) of the personal computing device may be further configured to process the stored biodata to facilitate a determination of the effectiveness of one or more ailment treatment.

### FIELD OF THE INVENTION

The present invention generally relates to chronic ailment treatments and evaluation tools that can accurately determine the effectiveness of one or more chronic ailment treatments. The effectiveness determinations may involve statistical analysis, datamining, and/or comparison of a substantial amount of an individual's biodata, obtained over a prolonged period of time. The biodata may be acquired directly from an afflicted individual or indirectly from someone else acting on their behalf, such as a family member or a personal medical care practitioner.

### BACKGROUND OF THE INVENTION

According to a recent study, roughly one in two Americans is already afflicted with a chronic medical condition of one type or another (at present, approximately 140 million Americans are afflicted). That number is projected to increase by more than one percent per year until the year 2030, resulting in an estimated chronically ill population of over 170 million people. (Chronic Care in America: A 21st Century Challenge, a study of the Robert Wood Johnson Foundation & Partnership for Solutions: Johns Hopkins University, Baltimore, Md. for the Robert Wood Johnson Foundation, September 2004 Update). Most of these afflicted individuals are in the early stages of their respective medical conditions and they are suffering from ailments that do not significantly impair their routine lifestyle activities. Accordingly, their adverse medical conditions may be effectively managed without significant risk or complications. However, if left untreated, a majority of these chronic medical conditions can rapidly progress into permanent, disabling maladies that render an individual unable to recover the quality of life they were accustomed to, prior to the onset of their deteriorated condition. Some epidemiologists researching this epidemic have predicted that at least half of the children alive in the U.S. today will eventually develop type-II diabetes.

Individuals and medical care providers in the United States alone spend billions of dollars per year on healthcare expenses related to the management and treatment of chronic ailments, including, but not limited to conditions such as, hypertension, hypercholesterolemia, hyperlipidemia, chronic obstructive pulmonary disease, diabetes mellitus, arthritis, chronic fatigue, migraine headaches, general anxiety disorder, obesity, and genetic risk of disease. In accordance with the Merriam Webster Online Dictionary (2009 ed.), an “ailment” is broadly defined as “a bodily disorder or chronic disease.” Accordingly, the terms: ailment, malady, illness, disease, and medical condition, are used interchangeably herein. These chronic medical conditions are increasingly being found in the U.S. and abroad as a result of increasingly poor dietary habits and other adverse lifestyle-related behaviors and activities that are associated with the use of many modern conveniences and consumables of the modern culture.

In the treatment of chronic ailments, a variety of name brand and generic medications are routinely being prescribed by health care providers as a sole treatment option or as a component of a prescribed treatment regimen. As would be understood by those skilled in the art, a generic drug or medication (also simply referred to as a “generic”) is a pharmaceutical treatment that is produced and distributed without patent coverage over the active ingredient(s). According to the U.S. Food and Drug Administration (FDA), generic medications are identical, or within an acceptable bioequivalent range, to their brand name counterpart with respect to pharmacokinetic and pharmacodynamic properties (relating to the drug's active ingredient). Accordingly, the FDA considers generics to be identical in dose, strength, route of administration, safety, efficacy, and intended use as their brand name counterparts. However, in many cases, as a result of lower quality manufacturing techniques (including utilizing less desirable inactive ingredients and bonding agents), generics are sometimes inferior to their brand name counterparts. In these cases, the question remains whether the quality differences between a generic and its brand name counterpart warrants the price difference between the two medications. Typically, several generics may become available after the patent protection afforded to the assignee/owner of the brand name medication has expired. Market competition may then lead to substantially lowered prices for both the brand name product and its generic spawn.

Unfortunately, the effectiveness of these various medication treatment options as well as complimentary dietary, exercise, and lifestyle treatment options are currently being evaluated for efficacy on the basis of a very limited set of medical practitioner obtained biodata. For example, a medication treatment may be prescribed or altered by a medical care provider based on a few blood pressure measurements acquired only while the patient was in the medical care provider's office during a routine or an emergency office visit. As would be understood by those skilled in the art, diagnosing ailments under these conditions, as well as prescribing and/or evaluating ailment treatments based on such a limited set of biodata, can often lead to inaccurate, misguided medical care. The worst case scenario is a misdiagnosis of a medical condition that results in a physician prescribing an improper treatment regimen to a patient that could negatively impact an existing chronic medical condition.

Clearly, it would be significantly more beneficial to have a larger pool of biodata, obtained over a prolonged period of time, with which to analyze a particular individual's medical condition and the progression of treatment options. It would therefore be desirable to have improved systems and methods that could advantageously utilize this large quantity of biodata in order to more accurately analyze the effectiveness of various chronic ailment treatments. Further, it would be beneficial if this analysis were offered to individuals within the comfort of their home or office and if personal biometric measurement devices along with personal computing devices were utilized to respectively acquire biodata used in the analysis of the treatments and then display treatment efficacy results. In this way, an individual could effectively self-monitor as well as routinely visualize the effectiveness of their particular ailment treatment(s) over both a short-term period of time (e.g., from day to day) or over an extended, long-term period of time (over the course of months or even years). This dual-term (short and long-term) analysis would improve treatment compliance and it could provide for a greater level of accuracy and an increased degree of precision for various treatment evaluation results.

It would also be desirable to utilize proven statistical analysis, datamining, and/or data comparison techniques to aid in the analysis of both empirically obtained and measured biodata, such that reliable evaluation techniques could be uniquely applied to the specialized field of medical treatment evaluation. In this way, more robust healthcare solutions could be offered to the ever-increasing population of chronically ill persons within the United States and abroad.

### SUMMARY OF THE INVENTION

This summary is provided to introduce (in a simplified form) a selection of concepts that are further described below in the Detailed Description. This summary is not intended to identify key features of the claimed subject matter, nor is it intended to be used as an aid in determining the scope of the claimed subject matter.

In overcoming the above disadvantages associated with modern systems and methods for chronic ailment management and treatment, the present invention includes an improved system that advantageously utilizes an afflicted individual's self monitoring ability in the analysis of one or more ailment treatments. In accordance with an embodiment of the invention, this system may include a personal computing device that has one or more processor, one or more memories, and a user interface. The system may further include one or more biometric measurement devices for acquiring biometric measurement data. In an embodiment, the personal computing device may be configured to receive various biodata and then store the biodata in memory. The processor(s) may be further configured to process the stored biodata to facilitate a determination of the effectiveness of one or more ailment treatments.

In accordance with another aspect of the present invention, the biodata may include biometric measurement data generated by one or more biometric measurement devices and/or personal health data obtained from a user input at the user interface of the personal computing device.

In accordance with a further aspect of the present invention, the personal health data may include: psychological health data, physiological health data, and/or psychobiological health data.

In accordance with yet another aspect of the present invention, the processor's processing of the stored biodata may include statistical analysis of the stored biodata.

In accordance with yet a further aspect of the present invention, the processor's processing of the stored biodata may include datamining the stored biodata.

In accordance with another aspect of the present invention, the one or more ailment treatments may be associated with a common ailment, including: hypertension, hypercholesterolemia, hyperlipidemia, chronic obstructive pulmonary disease, diabetes mellitus, arthritis, chronic fatigue, migraine headaches, general anxiety disorder, obesity, and/or genetic risk of disease.

In accordance with yet another aspect of the present invention, one or more biometric measurement devices may include: a blood pressure monitor, a blood glucose monitor, a blood lipid monitor, a blood ketone monitor, a blood oxygen saturation monitor, a urinalysis device, a bodyweight measurement device, a bodyfat measurement device, and/or a heartrate monitor.

In accordance with yet another aspect of the present invention, a computer-readable medium is encoded with a set of computer-executable instructions, which when executed, perform a method utilizing self-monitoring in the analysis of ailment treatments. The method may include: receiving a plurality of biodata at a personal computing device, storing the biodata, and processing the stored biodata to facilitate a determination of the effectiveness of at least one ailment treatment.

In accordance with yet another aspect of the present invention, a computer-implemented method may include: receiving a plurality of biodata at a personal computing device, storing the biodata, and processing the stored biodata to facilitate a determination of the effectiveness of at least one ailment treatment.

### DESCRIPTION OF THE DRAWINGS

Preferred and alternative examples of the present invention are described in detail below with reference to the following Figure drawings:

FIG. 1 illustrates a perspective view of a distributed computing system in accordance with an embodiment of the present invention;

FIG. 2 illustrates a perspective view of a Personal Computing Device in accordance with an embodiment of the present invention;

FIG. 3 illustrates a perspective view of a Biometric Measurement Device in accordance with an embodiment of the present invention;

FIG. 4 illustrates a flow diagram associated with an ailment treatment evaluation process in accordance with an embodiment of the present invention;

FIG. 5 illustrates a flow diagram associated with a user-interactive ailment treatment evaluation process in accordance with an embodiment of the present invention;

FIG. 6 illustrates a flow diagram associated with a hybrid ailment treatment evaluation process in accordance with an embodiment of the present invention;

FIG. 7 illustrates a flow diagram associated with a medication-based ailment treatment evaluation process in accordance with an embodiment of the present invention;

FIG. 8 illustrates a Personal Ailment Treatment Setup user interface associated with an embodiment of the present invention;

FIG. 9A-C illustrates various Personal Ailment Treatment healthcare dataforms associated with embodiments of the present invention;

FIG. 10 illustrates a Personal Ailment Treatment Input user interface associated with an embodiment of the present invention; and

FIG. 11 illustrates a Personal Ailment Treatment Summary user interface associated with an embodiment of the present invention.

### DESCRIPTION

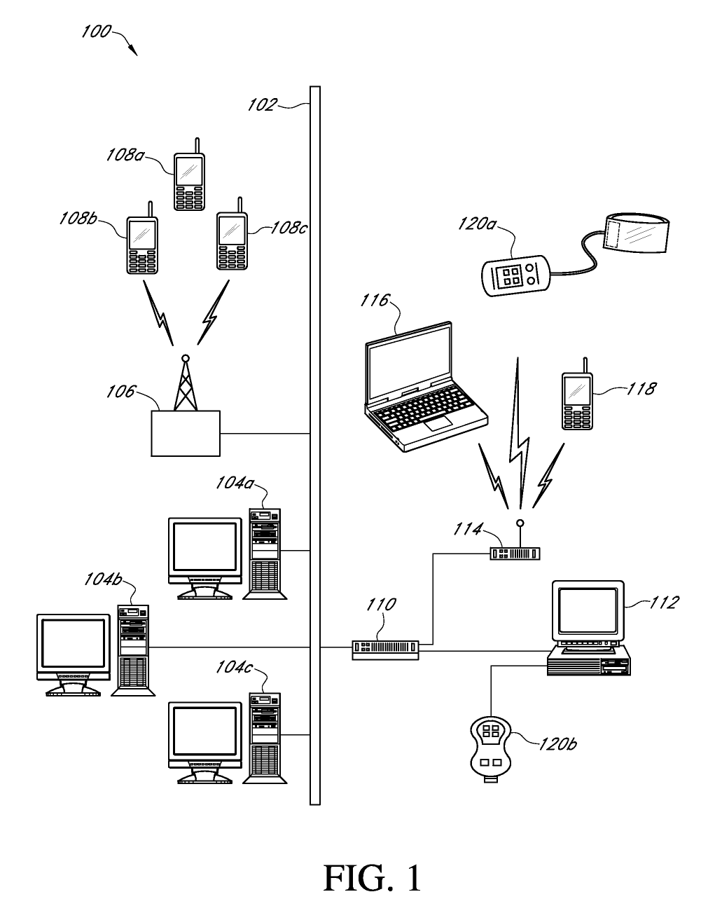

In accordance with an exemplary embodiment of the present invention, FIG. 1 illustrates a distributed computing system 100 including various wireline and wireless computing devices that may be utilized to implement any of the chronic ailment treatment processes associated with various embodiments of the present invention. The distributed computing system 100 may include, but is not limited to, one or more remote server computer devices 104a-c, any one of which may be associated with a remote Media Care Provider (MCP) that can provide medical care services and instructions to various networked clientele (operating one or more remote personal computing devices 108a-c, 112, 116, 118 and 120a-b); a communications network 102 (also referred to herein as a wide area network (WAN), including a wireless backhaul portion); one or more remote client devices 108a-c (e.g., cell phones, Personal Desktop Assistant (PDA) devices, and any other variety of common mobile computing device having biodata acquisition functionality) that may be connected to the WAN 102 utilizing a wireless basestation 106 or any common cable network technology (as part of the WAN backhaul); one or more network relay/gateway devices 110 that can facilitate various data communication processes between the WAN 102 and a local area network (LAN) portion of the distributed computing system 100. The LAN portion of the distributed computing system may include: a personal desktop computer 112 connected to one or more wireline biometric measurement devices 120b; a wireless router 114 that may communicate with various wireless LAN devices (116, 118, and 120a) using any common local wireless communications technology, such as Wi-Fi or unshielded twisted pair cable; a wireless laptop computer 116; a cell phone/PDA device 118; and one or more wireless biometric measurement devices 120a.

In an embodiment, the relay/gateway device 110 may also include local routing functionality to facilitate data communications amongst any LAN connected devices (112, 114, 116, 118, and 120a-b). In an embodiment, the remote server devices 104a-c, the wireless basestation 106, the remote client devices 108a-c, and any of the LAN connected devices (112, 116, 118, and 120a-b) may be configured to run any known operating system, including, but not limited to, Microsoft Windows™, Mac OS™, Linux™, Unix™, or any common mobile operating system, including Symbian™, Palm™, Windows Mobile™, Mobile Linux™, MXI™ etc. In an embodiment, the remote server devices 104a-c, the wireless basestation 106 may employ any number of common server, desktop, laptop, and personal computing devices.

In an embodiment, the remote client devices 108a-c and any of the LAN connected personal devices (112, 116, 118, and 120a-b) may include any combination of mobile computing devices and/or biometric measurement devices (e.g., cellular phones, PDAs, laptop computers, ultra-portable computers, mini-computers, as well as any variety of biometric measurement device, including but not limited to: blood pressure monitors, blood glucose monitors, blood lipid monitors, blood ketone monitors, blood oxygen saturation monitors, urinalysis devices, bodyweight measurement devices, bodyfat measurement devices, and heart rate monitors), having wireline or wireless communications capabilities. In an embodiment, some of these wireless communications capabilities may include the utilization of any common wireless data commutations protocol, such as GSM, UMTS, WiMAX, Wi-Fi, LTE protocols, or any other wireless communications technology known in the art. In an embodiment, some of the wireline communications capabilities may include the utilization of any common wireline communications technology, such as optical fiber, coaxial cable, twisted pair cable, Ethernet cable, power-line cable, or any other wireline communications technology known in the art.

In an embodiment, any of the remote server devices 104a-c, the wireless basestation 106, the remote client devices 108a-c, and any of the LAN connected devices (110, 112, 114, 116, 118 and 120a-b) may include any standard computing software and hardware necessary for processing, storing, and communicating data amongst each other within the distributed computing system 100. The high-level computing hardware may include, but is not limited to, one or more processors, volatile and non-volatile memories, user interfaces, biometric measurement units, transcoders, and wireline and/or wireless communications transceivers.

In an embodiment, a remote server device 104a-c (e.g., such as an MCP hosting a website-based treatment monitoring service) or any of the personal computing devices (108a-c, 112, 116, 118, and 120a-b, locally running treatment evaluation application software) may be configured to include a computer-readable medium (e.g., any common volatile or non-volatile memory type) encoded with a set of computer-readable instructions, which when executed, perform one or more biodata acquisition and treatment evaluation processes associated with the present invention. In either remote or local operation embodiments, data storage of user-acquired biodata (e.g., biometric measurement data and/or user determined healthcare data) may occur remotely or locally, on any variety of data storage medium well known in the art, without departing from the spirit and scope of the present invention (e.g., on a personal computer's or a server's resident hard drive, on an external hard drive, on a flash memory device, on a CD or DVD media, etc.). In one particular embodiment, any of the LAN connected devices (112, 114, 116, 118, and 120a-b) may be Wi-Fi Certified™ devices, capable of communicating amongst each other wirelessly.

As would be understood by those skilled in the art, the term “Wi-Fi” generally defines any wireless local area network (WLAN) operating in accordance with the Institute of Electrical and Electronics Engineers' (IEEE) 802.11 standards (e.g., 802.11(a), (b), and (n)). Wi-Fi is presently supported by most LAN devices, including personal desktop computers, laptops, minicomputers, PDAs and cellular phones, multi-function printers, biometric measurement devices, video game consoles and many other types of consumer home electronics. Wi-Fi Certified™ devices are typically interoperable with each other, even if they are from different manufacturers. For example, a user with a Wi-Fi Certified™ product can use any brand of access point with any other brand of client hardware that also is also Wi-Fi Certified™. For this reason, Wi-Fi communications of data content amongst LAN computing devices is ideal for short range transfer information at relatively high data transfer rates.

In another embodiment, any of the LAN connected devices (112, 114, 116, 118, and 120a-b) may be Bluetooth Certified™ devices, capable of communicating amongst each other wirelessly. As would be understood by those skilled in the art, Bluetooth Certified™ devices would need to communicate in much closer proximity than Wi-Fi Certified™ devices because of a relatively small communications coverage area.

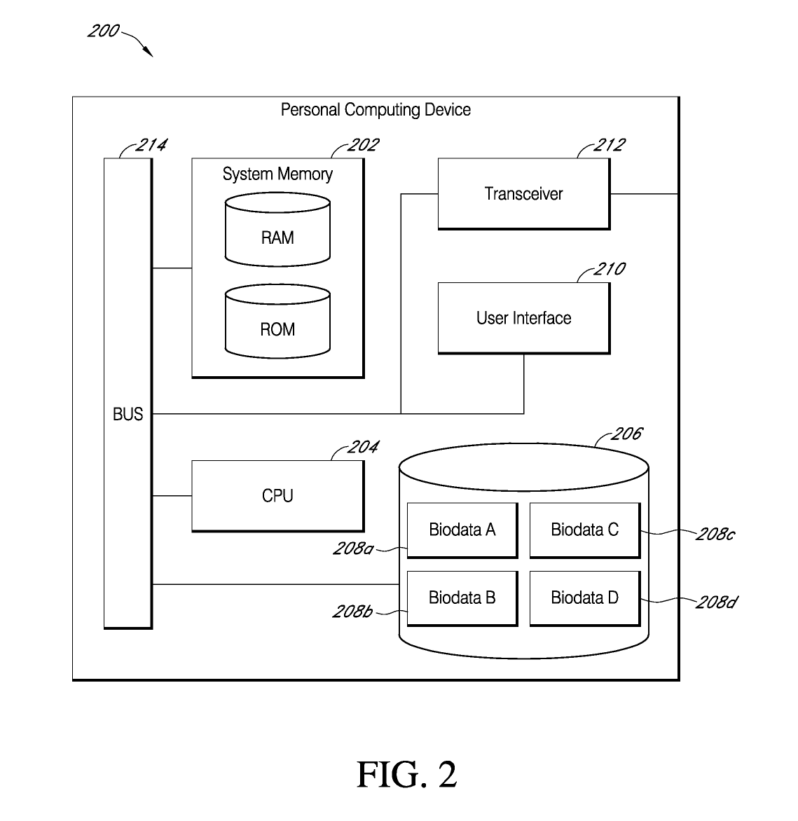

FIG. 2 shows a block diagram view of a Personal Computing Device (PCD) 200 that may be representative of any of the PCDs (108a-c, 112, 116, 118, or 120a-b) depicted in FIG. 1. The PCD 200 may include, but is not limited to, one or more processor devices including a central processing unit (CPU) 204. In an embodiment, the CPU 204 may include an arithmetic logic unit (ALU, not shown) that performs arithmetic and logical operations and one or more control units (CUs, not shown) that extract instructions and stored content from memory 202, 206 and then executes and/or processes them, calling on the ALU when necessary during program execution. The CPU 204 is primarily responsible for executing all computer programs stored on the PCD device's 200 volatile (RAM) and nonvolatile (ROM) system memories 202. The PCD device 200 may also include: a user interface 210 that facilitates a user's access to the PCD's 200 local resources; a content database 206 that includes repositories for storing various user-provided health data along with various acquired biometric measured data (both types of data referred to herein as “biodata”, e.g., Biodata repositories A-D 208a-d); and a transceiver 212 for transmitting and receiving data amongst the LAN connected devices (112, 116, 118, or 120a-b) and over the WAN 102 to any of the remote server devices 104a-c or the remote client devices 108a-c. In accordance with at least one embodiment of the present invention the PCD 200 may have a Wi-Fi Certified™ or a Bluetooth Certified™ multi-function transceiver device 212 that may be capable of communicating wirelessly with any LAN connected device (112, 116, 118, or 120a-b) that is also a Wi-Fi Certified™ or a Bluetooth Certified™ device.

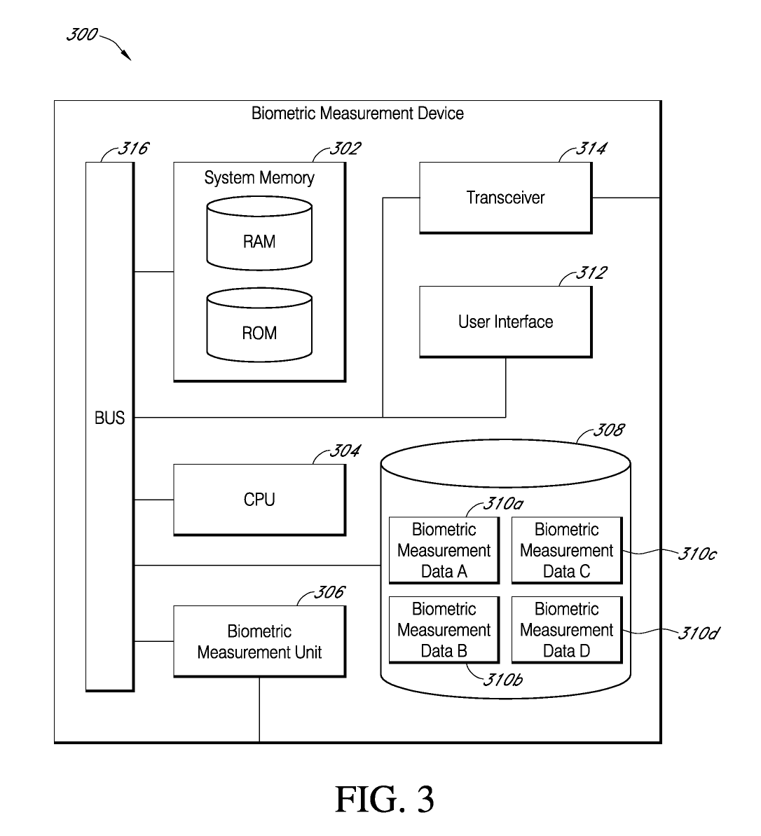

FIG. 3 shows a block diagram view of a Biometric Measurement Device (BMD) 300 that may be representative of any common wireless or wireline BMD (e.g., BMDs 120a-b of FIG. 1). In accordance with various embodiments of the present invention, the BMD 300 may also be associated with any of the following BMD devices: a blood pressure monitor, a blood glucose monitor, a blood lipid monitor, a blood ketone monitor, a blood oxygen saturation monitor, a urinalysis device, a bodyweight measurement device, a bodyfat measurement device, and a heartrate monitor. The BMD 300 may include, but is not limited to, one or more processor devices including a central processing unit (CPU) 304. The CPU 304 is responsible for executing all computer programs stored on the BMD device's 300 volatile (RAM) and nonvolatile (ROM) system memories 302 as well as controlling biometric measurement processes of the present invention. The BMD 300 may also include: a user interface 312 that facilitates a user's access to the BMD's 300 local resources; a content database 308 that includes repositories for storing various acquired biometric measurement data, interchangeably referred to herein as “biodata” (e.g., Biometric measurement data repositories A-D 310a-d); a biometric measurement unit 306 for measuring various user biodata, such as blood pressure, blood glucose, blood oxygen, etc.; and a transceiver 314 for transmitting and receiving data amongst the LAN connected devices (112, 116, 118, or 120a-b). In accordance with at least one embodiment of the present invention, the BMD 300 may have a Wi-Fi Certified™ or a Bluetooth Certified™ multi-function transceiver device 314 that may be capable of communicating wirelessly with any LAN connected device (112, 116, 118, or 120a-b) that is also a Wi-Fi Certified™ or a Bluetooth Certified™ device.

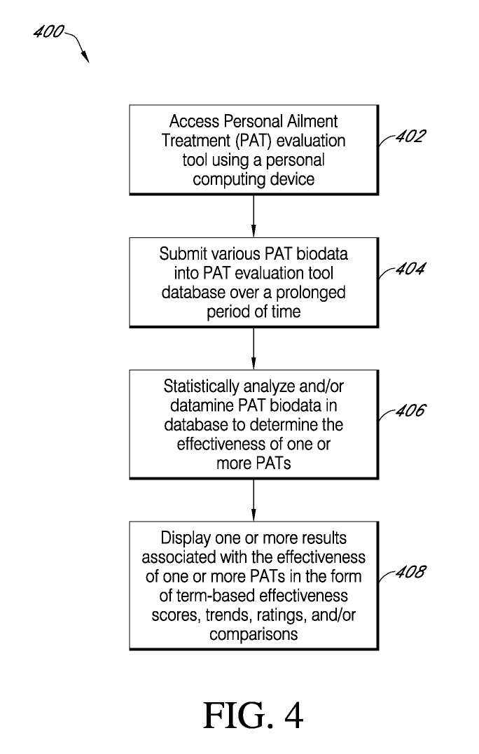

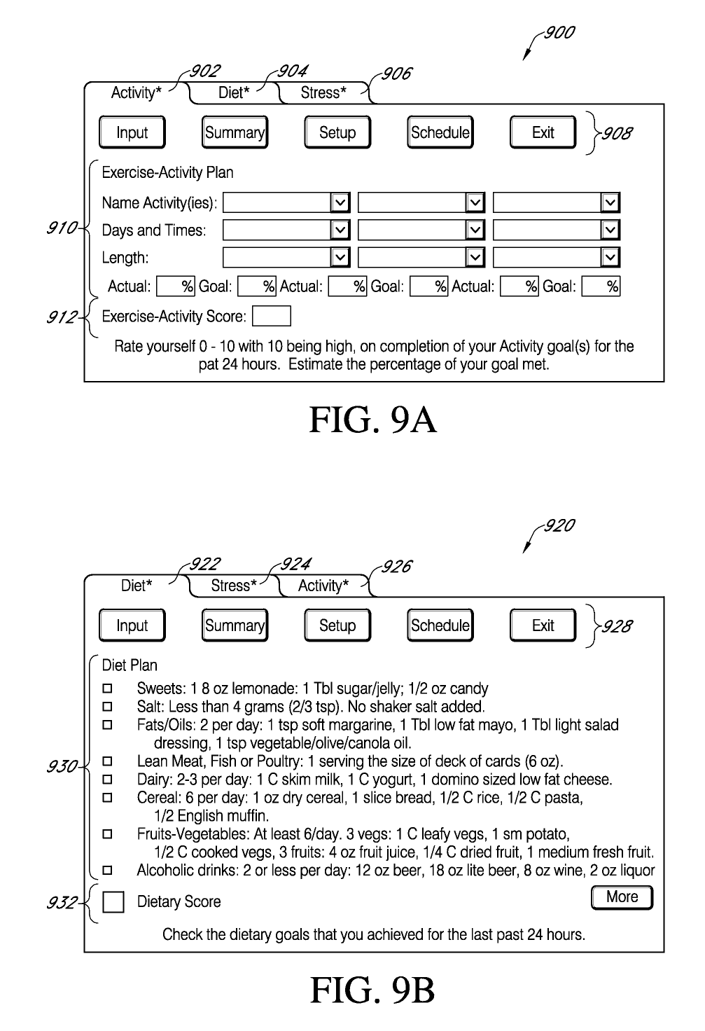

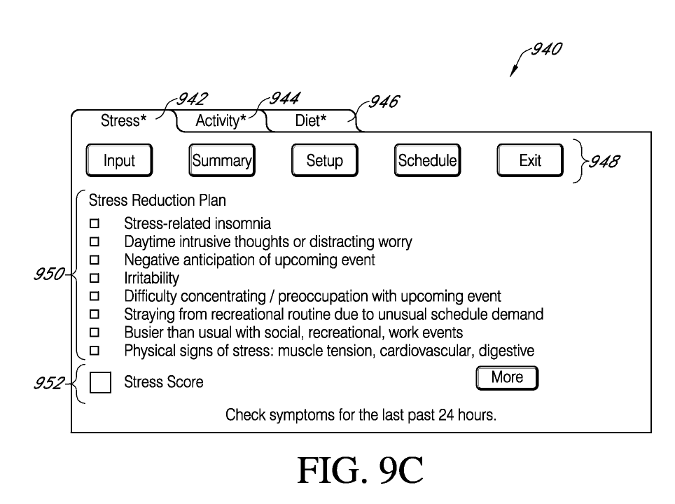

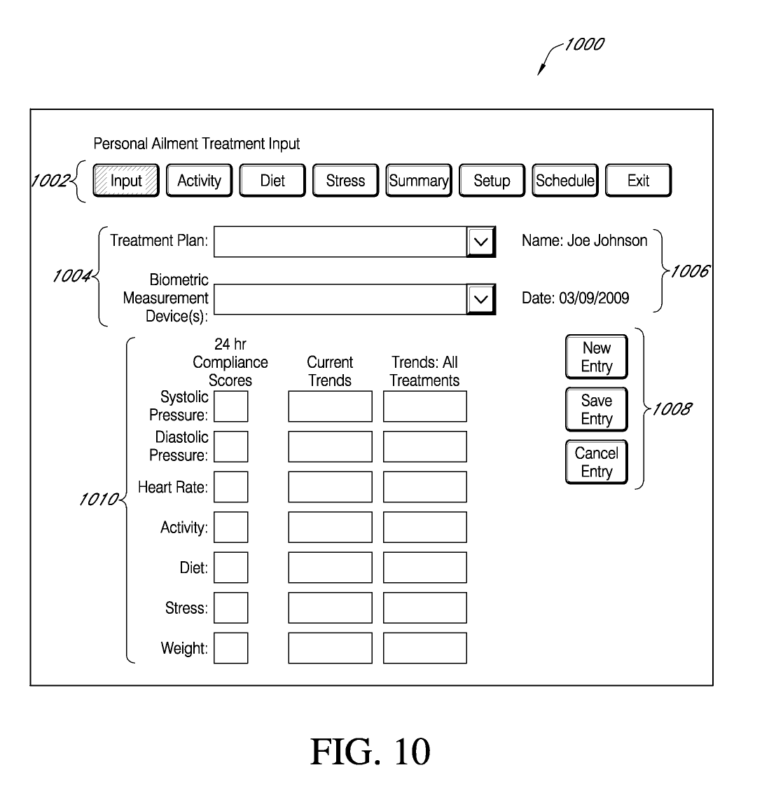

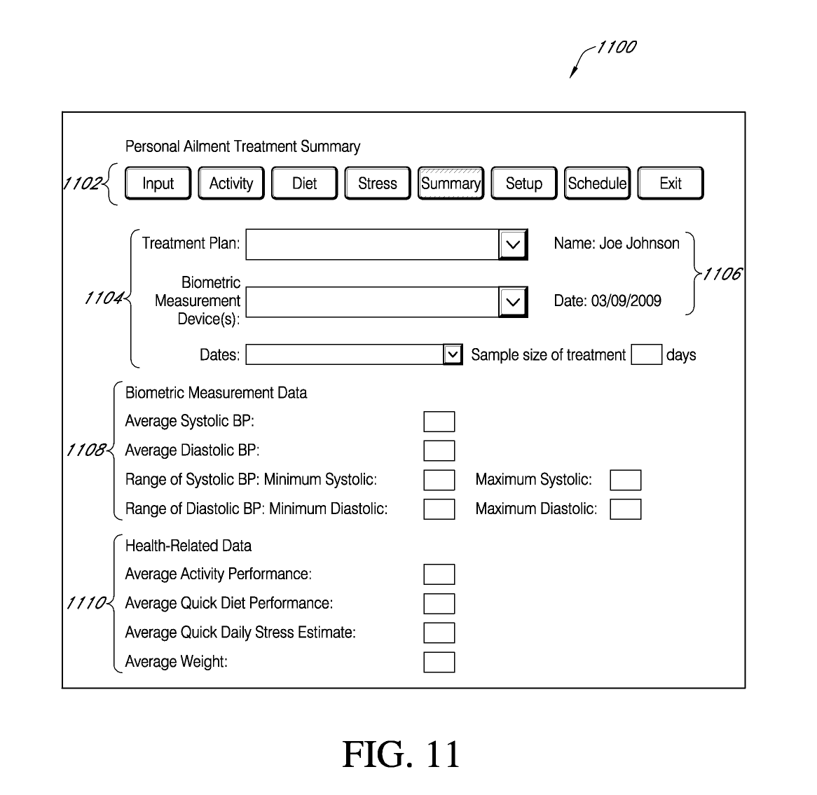

FIG. 4 illustrates a high-level flow diagram associated with a user-interactive ailment treatment evaluation process 400 in accordance with an embodiment of the present invention. It should be understood that this process 400 may be implemented by executing one or more computer-executable programs (e.g., a Personal Ailment Treatment (PAT) evaluation tool) stored on one or more computer-readable media located on any of the LAN connected personal computing devices (112, 116, 118, or 120a-b) or on any of the remote server devices 104a-c or PCDs 108a-c. At Block 402, a user accesses a PAT evaluation tool using a personal computing device (e.g., any personal computing devices 112, 116, 118, or 120a-b or 108a-c). Next at Block 404, a user submits various PAT biodata (e.g., any of user Biodata A-D 208a-d of FIG. 2, optionally including both biometric measurement data and general user healthcare data) into a PAT evaluation tool database (e.g., content database 206) over a prolonged period of time, using a PAT evaluation tool user interface (See e.g., user interfaces of FIGS. 9A-C and 10). In accordance with various embodiments of the invention, a prolonged period of time might be as short as a few days or as long as several years. Then at Block 406, the PAT evaluation tool may perform statistical analysis and/or datamining processes on the stored PAT Biodata (e.g., 208a-d) in order to determine the effectiveness of one or more PATs or PAT regimens. Finally, at Block 408, the PAT evaluation tool displays one or more results (See e.g., user interfaces of FIGS. 10-11) associated with the effectiveness of one or more PATs in the form of term-based effectiveness scores, trends, ratings, and/or comparisons.

As would be understood by those skilled in the art, statistics is a mathematical science pertaining to the collection, analysis, interpretation or explanation, and presentation of various data. Statistical analysis provides for analysis of obtained data as well as for prediction and forecasting of unknown data. Generally, samples of data (including biodata) representative of a larger population of data are obtained and subjected to statistical analysis, serving two related purposes: description and inference.

Descriptive statistics summarize a population of data by describing what was observed in the data samples numerically and/or graphically. Some common numerical descriptors include averages, means, standard deviations, and confidence intervals, while frequency and percentage are more useful in terms of describing categorical data.

Inferential statistics uses patterns in the sample data to draw inferences about the population of data being represented, accounting for randomness. These inferences may take the form of estimating numerical characteristics of the data, describing associations within the data (correlation), modeling relationships within the data (regression), extrapolation, interpolation, or other modeling techniques like analysis of variance (ANOVA), time series analysis, and data mining. In the context of the present invention, data mining is the process of extracting patterns from obtained samples of biodata. For both general statistical analysis and datamining processes associated with embodiments the present invention, it should be apparent that the larger the pool of sample biodata, the more accurate the PAT evaluation results will be.

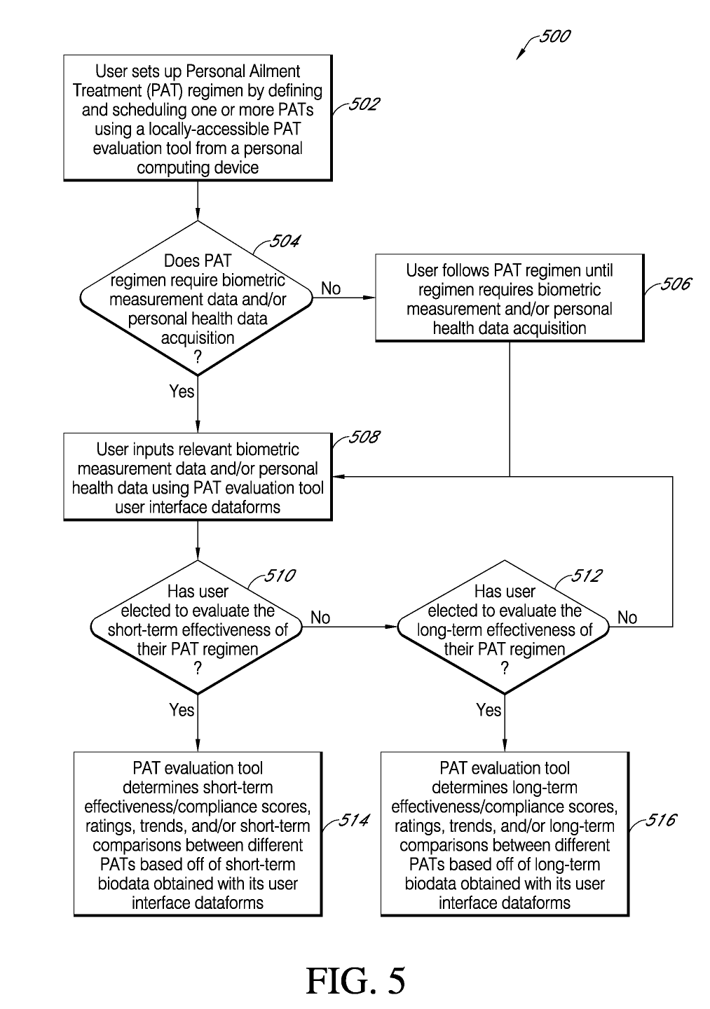

FIG. 5 illustrates a flow diagram associated with a user-interactive ailment treatment evaluation process 500 in accordance with an embodiment of the present invention. It should be understood that this process 500 may be implemented by executing one or more computer-executable programs (e.g., a Personal Ailment Treatment (PAT) evaluation tool) stored on one or more computer-readable media located on any of the LAN connected personal computing devices (112, 116, 118, or 120a-b) or on any of the remote server devices 104a-c or PCDs 108a-c. At Block 502, a user sets up a Personal Ailment Treatment (PAT) regimen by defining and scheduling one or more PATs using a locally-accessible PAT evaluation tool from a personal computing device (e.g., any of devices 112, 116, 118, 120a-b, or 108a-c). In accordance with various embodiments of the present invention, these PATs may be treatments associated with common ailments, including, but not limited to: hypertension, hypercholesterolemia, hyperlipidemia, chronic obstructive pulmonary disease, diabetes mellitus, arthritis, chronic fatigue, migraine headaches, general anxiety disorder, obesity, and/or genetic risk of disease. At decision Block 504, it is then determined if the PAT regimen requires biometric measurement data and/or personal health data acquisition. In accordance with various embodiments of the present invention, personal health data may include any psychological, physiological, and/or psychobiological health data relevant to a particular PAT. As would be understood by those skilled in the art, psychological health data would pertain to any health data relating to the mind or to mental phenomena as the subject matter of psychology. Physiological health data would pertain to any health data relating to the body or to organic processes or functions in a human body or in any of its parts as the subject matter of physiology. Psychobiological health data would pertain to the any health data relating to interactions between body and behavior as would be the subject matter of psychobiology. If biometric measurement data and/or personal health data acquisition is not required, at Block 506, a user continues to follow their present PAT regimen until that regimen does require biometric measurement and/or personal health data acquisition (at which point the process proceeds to Block 508). However, if biometric measurement data and/or personal health data acquisition is required, at Block 508, a user inputs all relevant/required biometric measurement data and/or personal health data using PAT evaluation tool user interface dataforms (See e.g., FIGS. 9A-C and 10).

Next at decision Block 510 it is determined if a user has elected to evaluate the short-term effectiveness of their PAT regimen. If the user has elected to evaluate the short-term effectiveness of their PAT regimen, at Block 514, the PAT evaluation tool determines short-term effectiveness/compliance scores, ratings, trends, and/or short term comparisons between different PATs based on short-term biodata obtained with its user interface dataforms. However, if the user has not elected to evaluate the short-term effectiveness of their PAT regimen, at decision Block 512, it is determined if the user has elected to evaluate the long-term effectiveness of their PAT regimen. If the user determined not to evaluate either the short-term or the long-term effectiveness of their PAT regimen, then the process reverts back to Block 508 (discussed supra). However, if the user determined to evaluate the long-term effectiveness of their PAT regimen, then at Block 516, the PAT evaluation tool determines the long-term effectiveness/compliance scores, ratings trends, and/or long-term comparisons between different PATs based on long-term biodata obtained with its user interface dataforms (See e.g., FIGS. 9A-C and 10). In accordance with an embodiment of the present invention, a user may also elect to evaluate both the short-term and the long-term effectiveness of their PAT regimen, such that separate or simultaneous display of effectiveness/compliance scores, ratings trends, and/or term-based comparisons between different PATs are facilitated respectively based on both short-term and long-term biodata obtained with its user interface dataforms (See e.g., FIGS. 9A-C and 10).

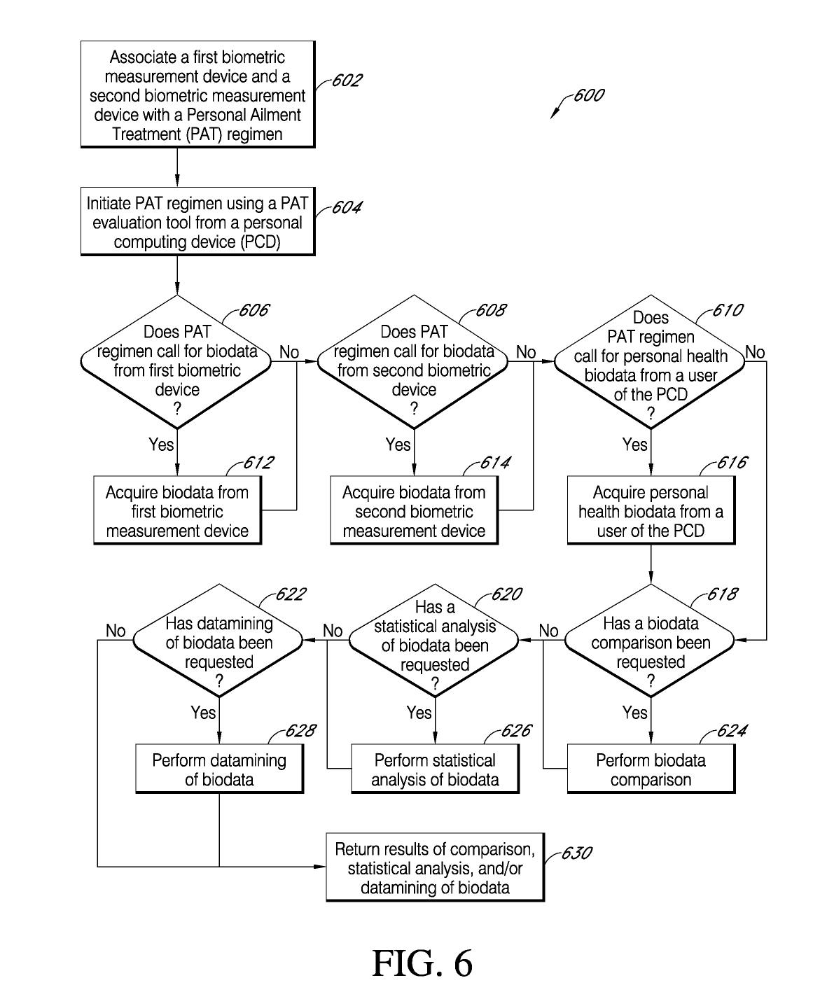

FIG. 6 illustrates a flow diagram associated with a hybrid ailment treatment evaluation process 600 including the utilization of multiple biodata types and multiple evaluation techniques in accordance with an embodiment of the present invention. It should be understood that this process 600 could be implemented by executing one or more computer-executable programs (e.g., a Personal Ailment Treatment (PAT) evaluation tool) stored on one or more computer-readable media located on any of the LAN connected personal computing devices (112, 116, 118, or 120a-b) or on any of the remote server 104a-c or personal computing devices 108a-c. At Block 602, both a first (e.g., 120a) and a second (e.g., 120b) biometric measurement device are respectively associated with one or more defined PAT regimens. Next at block 604, at least one of the associated PAT regimens is initiated using a PAT evaluation tool, from a personal computing device (e.g., any of 108a-c, 112, 116, 118, or 120a-b). Then at decision block 606, it is determined if the initiated PAT regimen calls for biodata from the first biometric measurement device. If the PAT regimen does call for biodata from the first biometric measurement device, then at block 612, the first biometric measurement device is utilized to acquire the required biodata (e.g., blood pressure biodata). Subsequently, the process proceeds to decision block 608.

However, if the PAT regimen does not call for biodata from the first biometric measurement device (e.g., 120a), then at decision block 608, it is determined if the initiated PAT regimen calls for biodata from the second biometric measurement device (e.g., 120b). If the PAT regimen does call for biodata from the second biometric measurement device, then at block 614, the second biometric measurement device is utilized to acquire the required biodata (e.g., blood glucose biodata). Subsequently, the process proceeds to decision block 610. However, if the PAT regimen does not call for biodata from the second biometric measurement device, then at decision block 610, it is determined if the initiated PAT regimen calls for personal health biodata from a user of the PCD (e.g., any of 108a-c, 112, 116, 118, or 120a-b).

If the PAT regimen does call for personal health biodata from a user of the PCD, then at block 616, the user of the PCD submits the required personal health biodata (e.g., dietary, exercise, and stress related biodata) utilizing a user interface of a PAT evaluation tool (e.g., by using any of the user interfaces depicted in FIGS. 9A-C and 10). Subsequently, the process proceeds to decision block 618. However, if the PAT regimen does not call for personal health biodata, then at decision block 618, it is determined if a biodata comparison has been requested. If a biodata comparison has been requested, then at block 624, the biodata comparison is performed (e.g., a comparison of biodata for the same PAT during different time periods or a comparison of biodata for different PATs during the same and/or different time periods). Subsequently, the process proceeds to decision block 620.

However, if a biodata comparison has not been requested, at decision block 620, it is determined if a statistical analysis of biodata has been requested. If a statistical analysis of biodata has been requested, then at block 626 the statistical analysis of the biodata is performed. Subsequently, the process proceeds to decision block 622. However, if a statistical analysis of the biodata has not been requested, then at decision block 622 it is determined if a datamining of the biodata has been requested. If a datamining of the biodata has been requested, then at block 628, a datamining of the biodata is performed. Subsequently, the process proceeds to block 630. However, if a datamining of the biodata has not been requested, then the process proceeds to block 630. At block 630, the results of any requested data processing, including biodata comparison, statistical analysis of biodata, and/or datamining of biodata is returned to the requesting user as one or more evaluation results (See e.g., PAT results summary form 1100 of FIG. 11).

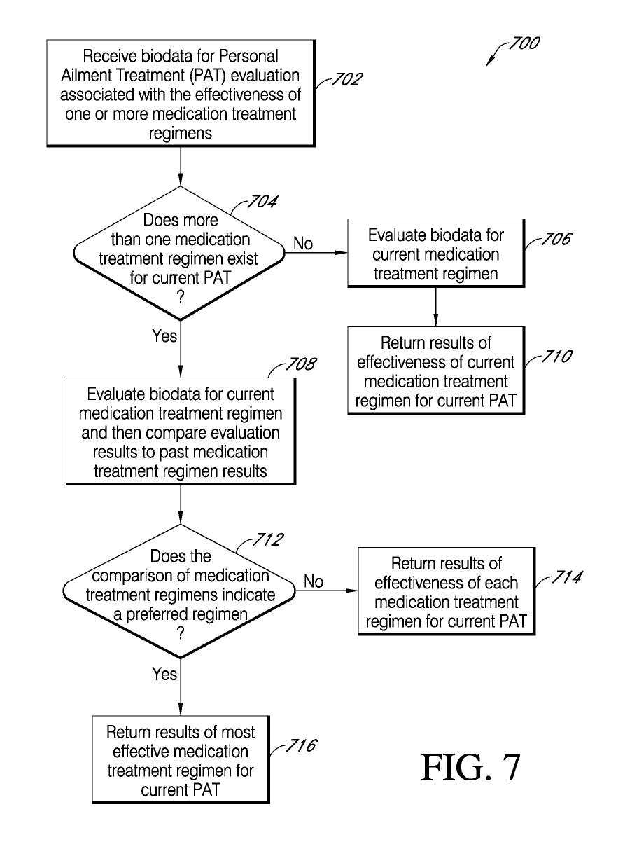

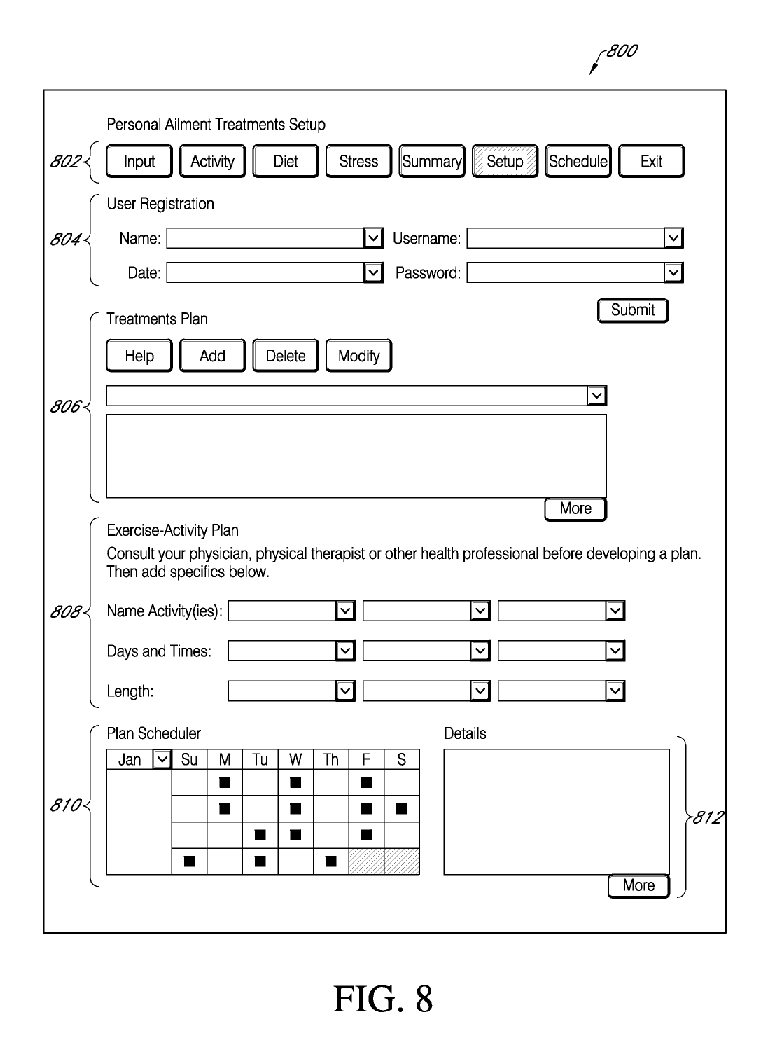

FIG. 7 illustrates a flow diagram associated with a medication-based ailment treatment evaluation process 700 in accordance with an embodiment of the present invention. It should be understood that this process 700 may be implemented by executing one or more computer-executable programs (e.g., a Personal Ailment Treatment (PAT) evaluation tool) stored on one or more computer-readable media located on any of the LAN connected personal computing devices (112, 116, 118, or 120a-b) or on any of the remote server devices 104a-c or PCDs 108a-c. At Block 702, biodata associated with the effectiveness of one or more medication treatment regimens is received at a Personal Ailment Treatment (PAT) evaluation tool (e.g., utilizing any of the PAT user interfaces depicted in FIG. 8 or 10). Next at decision block 704, it is determined if one or more medication treatment regimens exist for the current PAT (e.g., there could be a generic and brand name medication treatment (e.g., medication having the same active ingredient(s)) regimen for the same PAT that would be implemented at different times, or there could be different medications treatment regimens (e.g., medications having different active ingredient(s)) used at the same or different times for the same PAT. If only one medication treatment regimen existed for the current PAT, then at block 706, biodata for the current, single medication treatment regimen would be evaluated, and then at block 710, results depicting the effectiveness of a current medication treatment regimen for the current PAT would be returned and displayed to a user (e.g., utilizing PAT results summary of FIG. 11).

However, if multiple medication treatment regimens existed for a current PAT, then at block 708, biodata for a current medication treatment regimen would be evaluated and then compared to existing evaluation results for past selected medication treatment regimens (e.g., comparing the efficacy of two different types of blood pressure medications). Next at decision block 712, it would be determined if the comparison of medication treatment regimens indicated a preferred PAT regimen (e.g., when one medication had superior biodata results and fewer health related side effects). If the comparisons of the medication treatment regimens indicated no significantly preferred medication treatment regimen, then at block 714, the efficacy results of each medication treatment regimen for the current PAT would be returned and displayed to a user. However, if the comparisons of the medication treatment regimens did reveal a superior preferred medication treatment regimen, then at block 716, the efficacy results of only the preferred, most effective medication treatment regimen for the current PAT would be returned and displayed to a user.

FIG. 8 illustrates a Personal Ailment Treatment (PAT) Setup interface that could be associated with a server-based PAT website (a MCP website running on any or remote server devices 104a-c) or a stand-alone PAT computer application running from a user's personal computing device (e.g., running from any of PCDs 108a-c, 112, 116, 118, or 120a-b). In an embodiment, the interface 800 may be configured to include a menu 802 of tabs or buttons that link or hyperlink to various embedded application interfaces or dataforms when the tabs or buttons are selected (e.g., clicked). In accordance with an embodiment, the menu 802 may include tabs or buttons linking to: “Input”, “Activity”, “Diet”, “Stress”, “Summary”, and “Schedule” interfaces. In an embodiment of a server-based PAT website (a MCP website running on any of remote server devices 104a-c), these interfaces or dataforms may represent webpages of a PAT website. In an embodiment of a stand-alone PAT computer application running from a user's personal computing device, these interfaces or dataforms may represent separate interface forms stored locally in PCD memory or on an external harddrive.

In an embodiment, the interface 800 may be further adapted to include a user registration section 804, where a user is able to enter various registration and/or login information including, but not limited to, their Name, the current Date, their Username and Password, or any other common access identification information known in the Art; a Treatment Plan entry section 806 where a user can select, add, delete, or modify any prescribed PAT regimen stored in a PAT database. In an embodiment, a listing of available PATs is selectable from a dropdown menu and the available PATs correspond to treatments uploaded by a medical care provider, such as a primary physician. In various embodiments, these treatments may correspond to PATs associated with one or more of the following ailments: hypertension, hypercholesterolemia, hyperlipidemia, chronic obstructive pulmonary disease, diabetes mellitus, arthritis, chronic fatigue, migraine headaches, general anxiety disorder, obesity, and genetic risk of disease; An Exercise Activity Plan section 808, where a user can select, multiple exercise activity types and associated scheduling times and durations for each exercise activity; and a Plan Scheduler 810 where a user can access a calendar-based scheduling interface to coordinate their various treatment plan activities. In an embodiment the interface 800 may also include a Plan Scheduler details section 812 that displays highlighted (e.g., scrolled over calendar dates) plan details for any particular day(s) or week(s) of a scheduled month.

FIGS. 9A-C illustrate an interface of a tabbed group of PAT dataforms 900, 920, and 940, associated with the “Activity”, “Diet”, and “Stress” buttons of the PAT interface 800 depicted in FIG. 8. In accordance with an alternate embodiment, these tabbed dataforms 900, 920, and 940, may also be represented as cascading forms arrayed such that one form is displayed on top of the other (e.g., form 900 in front of form 920, which itself is in front of form 940). In an embodiment, the Activity form 900 of FIG. 9A may have a forward Activity file-tab 902, as well as rearward Diet 904 and Stress 906 file-tabs that respectively display corresponding PAT regimen components when selected (e.g., by a user clicking on a tab). Similar to FIG. 8, the Activity form 900 may include a menu 908 of tabs or buttons that link or hyperlink to various embedded application interfaces when the tabs or buttons are selected (e.g., clicked). In accordance with an embodiment, the menu 908 may include tabs or buttons linking to: “Input”, “Summary”, “Setup”, and “Schedule” interfaces. The Activity form 900 may also include an Exercise Activity Plan section 910, where a user can select, multiple exercise activity types and associated scheduling times and durations for each selected exercise activity. Further, the Exercise Activity Plan section 910, may also include areas for displaying individual activity compliance percentages associated with actual exercise activity compliance and an exercise activity compliance goal, displaying a desired exercise activity compliance. The Activity form 900 may also include a total exercise activity score section 912, which may be representative of an aggregate actual compliance score for all scheduled exercise activities.

In an embodiment, the Diet form 920 of FIG. 9B may have a forward Diet file-tab 922, as well as rearward Stress 924 and Activity 926 file-tabs that respectively display corresponding PAT regimen components when selected (e.g., by a user clicking on a tab). The Diet form 920 may include a menu 928 of tabs or buttons that link or hyperlink to various embedded application interfaces when the tabs or buttons are selected (e.g., clicked). In accordance with an embodiment, the menu 928 may include tabs or buttons linking to: “Input”, “Summary”, “Setup”, and “Schedule” interfaces. The Diet form 920 may also include a dietary goals section 930 that facilitates a user selecting multiple term-based dietary goals (e.g., dietary consumption goals for a 24 hour period). Further, the Diet form 920 may also include a total dietary score section 932, which may be representative of an aggregate dietary score for a predetermined period of time.

In an embodiment, the Stress form 940 of FIG. 9C may have a forward Stress file-tab 942, as well as rearward Activity 944 and Diet 946 file-tabs that respectively display corresponding PAT regimen components when selected (e.g., by a user clicking on a tab). The Stress form 940 may include a menu 948 of tabs or buttons that link or hyperlink to various embedded application interfaces when the tabs or buttons are selected (e.g., clicked). In accordance with an embodiment, the menu 948 may include tabs or buttons linking to: “Input”, “Summary”, “Setup”, and “Schedule” interfaces. The Stress form 940 may also include a section 950 that facilitates a user selecting multiple stress related symptoms that could facilitate a determination of a proximate level of stress an individual is experiencing over short-term and long-term periods of time. Further, the Stress form 940 may also include a total stress score section 952, which may be representative of an aggregate stress score for a predetermined period of time.

FIG. 10 illustrates a Personal Ailment Treatment (PAT) Input interface 1000 that may be associated with a server-based PAT website or a stand-alone PAT computer application running from a user's personal computing device. In an embodiment, the interface 1000 may be configured to include a menu 1002 of tabs or buttons that link or hyperlink to various embedded application interfaces or dataforms when the tabs or buttons are selected (e.g., clicked). In accordance with an embodiment, the menu 802 may include tabs or buttons linking to: “Activity”, “Diet”, “Stress”, “Summary”, “Setup” and “Schedule” interfaces.

In an embodiment, the PAT Input interface 1000 may include a Treatment Plan and Biometric Measurement Device selection component 1004 that may facilitate a registered user inputting and/or selecting various ailment treatment plans as well as registering and/or selecting various available biometric measuring devices available to a user that is utilizing one or more ailment treatments or regimens. The PAT Input interface 1000 may also include a display component 1006, indicating an active user along with the present date and time. The Input interface 1000 may further be adapted to include a treatment biodata input/display section 1010 that may allow for input and/or display of selected biometric measurement results (e.g., biodata acquired from select biometric measurement devices) as well as general health related data results (e.g., biodata results imported from Activity 900, Diet 920, and Stress 940 dataforms of FIGS. 9A-C). In an embodiment, the treatment biodata input/display section 1010 may also include current trends results for single and multiple treatments, to facilitate a user's determination of one or more health trends related to obtained biodata. In an embodiment, the entry and modification of various treatment biodata results may be facilitated by an entry modification menu component 1008 that may include buttons facilitating new biodata results entries, as well as the options to save or cancel biodata results entries.

FIG. 11 illustrates a Personal Ailment Treatment (PAT) Summary interface 1100 that may be associated with a server-based PAT website or a stand-alone PAT computer application running from a user's personal computing device. In an embodiment, the interface 1100 may be configured to include a menu 1102 of tabs or buttons that link or hyperlink to various embedded application interfaces or dataforms when the tabs or buttons are selected (e.g., clicked). In accordance with an embodiment, the menu 1104 may include tabs or buttons linking to: “Input”, “Activity”, “Diet”, “Stress”, “Setup” and “Schedule” interfaces.

In an embodiment, the PAT Summary interface 1100 may include a Treatment Plan and Biometric Measurement Device selection component 1104 that may facilitate a registered user inputting and/or selecting various ailment treatment plans as well as selecting various biometric measuring devices affiliated with the ailment treatment plans. This selection may facilitate an evaluation of select biodata for one or more treatment plans utilizing one or more biometric measurement devices. In an embodiment, the Treatment Plan and Biometric Measurement Device selection component 1104 may further facilitate a user's selection of a date range and/or a biodata sample size with which to make various PAT evaluations. For example, a user may wish to evaluate blood pressure, activity, diet, stress, and bodyweight biodata over a period of a 30 days for a select date range (e.g., January 12 through February 10). Based on a user's selection of one or more PATs and affiliated biodata, an evaluation based on statistical analysis, datamining, and/or comparison may be performed and the corresponding results may be displayed in a Biometric Measurement Data 1108 and a Health Related Data 1110 results summary sections of the PAT Summary interface 1100.

In accordance with various other embodiments of the present invention, the PAT evaluation tool interfaces depicted in FIGS. 8-11 may be modified for particular ailment treatments or PAT regimens (e.g., customized interfaces for Blood Pressure Management, Diabetes Management, Cholesterol Management, Migraine Headache Management, etc.), such that the components and contents of the respective interfaces, and any underlying program code, included information, data fields, and functionality to effectively evaluate PAT's affiliated with specific ailments.

The present invention provides for a truly dynamic means for individuals and patients to monitor their own health status, whether they are self-managing their own PAT regimen or managing a prescribed PAT regimen under the supervision of a medical care provider. The invention allows an individual to become an active agent in their own healthcare management plan as they gather and store large samples of biodata using personal computing devices in their home or office. Now that modern technologies allow individuals to acquire and store significant quantities of personal biodata from home, the use of scientific statistical methods in the analysis of ailment treatments is no longer limited to medical and scientific researchers. Accordingly, the present invention takes advantage of these new technologies and advantageously combines its functionality with highly accurate scientific statistical methods to allow the chronically ill population to proactively manage their own health.

By way of example, a user of the present invention may analyze personal biodata using the PAT evaluation tool (e.g., partially depicted in FIGS. 8-11) to learn that it does not matter whether his hypertension medication is a brand name or a generic as long as he keeps his weight under 200 pounds and maintains a dietary compliance rating of 70 percent or above. Prior to the present invention, these complex relationships between ailment treatments and their measured biodata could not be determined through even routine office visits to a primary care physician. Accordingly, the present invention acts as a practical improvement and a costs-benefit analysis tool in the treatment of modern-day chronic ailments. As technology advances and more and more affordable home biometric measurement devices become available to the public, the present invention will become even more effective and necessary to reduce professional health care related expenses.

Prior to the present invention there were no practical solutions available to practicing medical care practitioners for accurately evaluating ailment treatment effectiveness for individual patients. Rather, treatment decisions were typically made on the basis of published medical research rather than on the observed effectiveness of a given treatment on the individual patient actually being treated in a medical care practitioner's office. To a great extent, medical care practitioners have had to rely on a “best guess” of how their patients should react to a medication rather than on the basis of how a patient actually reacts to a specific treatment. Medical checkups are often infrequent due to the cost of office visits. Where individuals are capable of recording their own biometric data and capable of presenting their data during a medical care practitioner office visit, practitioners are often not properly trained or reimbursed for the statistical analysis of collected data. Out of a need to protect the patient, medical care practitioners tend to prescribe the most expensive treatments. As the cost of medication for a specific disease can vary ten-fold, there is clearly a financial justification for filling this gap in medical technology. The current invention meets this need by providing for a solution for collecting and analyzing large amounts of medical data collected not in clinic but at home or in the workplace.

The current invention can reduce the need for medical office visits thereby reducing healthcare costs to the individual and to society in general. The current invention contains an electronic medium and thereby opens the door for changes in medical office practices that may prove cost-effective over time. This may in turn reduce the need for expensive and time consuming medical office visits. The medical risks of trying generic or inexpensive medications may be reduced as monitoring and/or analysis can be performed daily and safely using existing personal medical measurement devices.

In accordance with the present invention, an individual patient can test their own ability to contribute to improving their health by the inclusion of life style improvements and to have the effectiveness of a progression of life style changes on the health condition. This may reduce costs and improve the general health of the individual. Individuals may choose to increase the intensity of their diet and exercise program before moving on to a more expensive medication or treatment through use of the invention. The user can examine an individualized treatment analysis and further increase compliance when their level of compliance has room to improve. These features of the invention may significantly reduce the need for office visits and average health care costs.

The invention can lend itself to the multivariate analysis of medication effects for the purposes of scientific testing and development of new treatments and medications. The feature of the program can generate valuable medical data as individual patients donate their health database to medical research institutions.

American healthcare has long been criticized for its lack of emphasis on primary care/preventive strategies. Most physicians recommend only medications with only a passing encouragement of have a healthy lifestyle. The current invention can allow for scientific analysis of the effects of lifestyle issues on the effects of a prescribed medication.

While several embodiments of the present invention have been illustrated and described herein, many changes can be made without departing from the spirit and scope of the invention. Software products associated with the computer-readable media of the present invention could conceivably be obtained from a retail store, a medical care facility, or over the internet (as a downloadable file) as a stand-alone software utility or as a component of a larger software utility, such as a plug-in or add-on component to an existing software utility. It should be understood that the spirit and scope of the invention is not limited by any one disclosed embodiment. Instead, the scope of the invention should be determined from the appended claims that follow.

The embodiments of the invention in which an exclusive property or privilege is claimed are defined as follows:

1. A system for utilizing self-monitoring in the analysis of ailment treatments, the system comprising:
a personal computing device comprising,
at least one processor;
at least one memory; and
a user interface; and
at least one biometric measurement device,
wherein the personal computing device is configured to receive a plurality of biodata and then store the plurality of biodata in memory, and
wherein at least one processor is configured to process the stored plurality of biodata to facilitate a determination of the effectiveness of at least one ailment treatment.

2. The system of claim 1, wherein the plurality of biodata comprises biometric measurement data generated by at least one biometric measurement device and personal health data obtained from user input at the user interface of the personal computing device.

3. The system of claim 2, wherein personal health data are selected from the group consisting of: psychological health data, physiological health data, and psychobiological health data.

4. The system of claim 1, wherein at least one processor's processing of the stored plurality of biodata further comprises statistical analysis of the stored plurality of biodata.

5. The system of claim 1, wherein at least one processor's processing of the stored plurality of biodata further comprises datamining of the stored plurality of biodata.

6. The system of claim 1, wherein at least one ailment treatment is associated with a common ailment selected from the group consisting of: hypertension, hypercholesterolemia, hyperlipidemia, chronic obstructive pulmonary disease, diabetes mellitus, arthritis, chronic fatigue, migraine headaches, general anxiety disorder, obesity, and genetic risk of disease.

7. The system of claim 1, wherein at least one biometric measurement device is selected from the group consisting of: a blood pressure monitor, a blood glucose monitor, a blood lipid monitor, a blood ketone monitor, a blood oxygen saturation monitor, a urinalysis device, a bodyweight measurement device, a bodyfat measurement device, and a heartrate monitor.

8. A computer-readable medium encoded with a set of computer-executable instructions, which when executed, perform a method for utilizing self-monitoring in the analysis of ailment treatments, the method comprising:
receiving a plurality of biodata at a personal computing device;
storing the plurality of biodata; and
processing the stored plurality of biodata to facilitate a determination of the effectiveness of at least one ailment treatment.

9. The computer-readable medium of claim 8, wherein the plurality of biodata comprises biometric measurement data generated by least one biometric measurement device and personal health data obtained from a user of the personal computing device.

10. The computer-readable medium of claim 9, wherein the personal health data are selected from the group consisting of: psychological health data, physiological health data, and psychobiological health data.

11. The computer-readable medium of claim 8, wherein the processing of the stored plurality of biodata further comprises statistical analysis of the stored plurality of biodata.

12. The computer-readable medium of claim 8, wherein the processing of the stored plurality of biodata further comprises datamining of the stored plurality of biodata.

13. The computer-readable medium of claim 8, wherein at least one ailment treatment is associated with a common ailment selected from the group consisting of: hypertension, hypercholesterolemia, hyperlipidemia, chronic obstructive pulmonary disease, diabetes mellitus, arthritis, chronic fatigue, migraine headaches, general anxiety disorder, obesity, and genetic risk of disease.

14. The computer-readable medium of claim 8, wherein the plurality of biodata comprise biometric measurement data generated by at least one biometric measurement device that is selected from the group consisting of: a blood pressure monitor, a blood glucose monitor, a blood lipid monitor, a blood ketone monitor, a blood oxygen saturation monitor, a urinalysis device, a bodyweight measurement device, a bodyfat measurement device, and a heartrate monitor.

15. A computer-implemented method for utilizing self-monitoring in the analysis of ailment treatments, the method comprising:
receiving a plurality of biodata at a personal computing device;
storing the plurality of biodata; and
processing the stored plurality of biodata to facilitate a determination of the effectiveness of at least one ailment treatment.

16. The computer-implemented method of claim 15, wherein the plurality of biodata comprise biometric measurement data generated by at least one biometric measurement device and personal health data obtained from a user of the personal computing device.

17. The computer-implemented method of claim 16, wherein the personal health data are selected from the group consisting of: psychological health data, physiological health data, and psychobiological health data.

18. The computer-implemented method of claim 15, wherein the processing of the stored plurality of biodata further comprises statistical analysis and datamining of the stored plurality of biodata.

19. The computer-implemented method of claim 15, wherein at least one ailment treatment is associated with a common ailment selected from the group consisting of: hypertension, hypercholesterolemia, hyperlipidemia, chronic obstructive pulmonary disease, diabetes mellitus, arthritis, chronic fatigue, migraine headaches, general anxiety disorder, obesity, and genetic risk of disease.

20. The computer-implemented method of claim 15, wherein the plurality of biodata comprise biometric measurement data generated by at least one biometric measurement device that is selected from the group consisting of: a blood pressure monitor, a blood glucose monitor, a blood lipid monitor, a blood ketone monitor, a blood oxygen saturation monitor, a urinalysis device, a bodyweight measurement device, a bodyfat measurement device, and a heartrate monitor.
